(19) United States
(12) Patent Application Publication
(10) Pub. No.: US 2018/0305754 A1
(43) Pub. Date: Oct. 25, 2018

(54) BIOMARKER FOR DIAGNOSIS OF DRUG ADDICTION AND KIT FOR DIAGNOSIS OF DRUG ADDICTION

(71) Applicant: KOREA INSTITURE OF SCIENCE AND TECHNOLOGY, Seoul (KR)

(72) Inventor: Heh-In IM, Seoul (KR)

(73) Assignee: KOREA INSTITURE OF SCIENCE AND TECHNOLOGY, Seoul (KR)

(21) Appl. No.: 15/743,836

(22) PCT Filed: Jul. 12, 2016
(86) PCT No.: PCT/KR2016/007560

§ 371 Date: Jan. 11, 2018

(30) Foreign Application Priority Data
Jul. 14, 2015 (KR) 10-2015-0099743

(51) Int. Cl. C12Q 1/6876; G01N 33/68

(52) U.S. Cl. CPC C12Q 1/6876; G01N 33/6893; G01N 2800/307; C12Q 2600/158; C12Q 2600/178; C12Q 2600/112

(57) ABSTRACT

The present disclosure relates to a biomarker for diagnosis of drug addiction and a kit for diagnosis of drug addiction. The biomarker for diagnosis of drug addiction and the kit for diagnosis of drug addiction according to the present disclosure can determine drug addiction by using a molecular biological method. Especially, the present disclosure has investigated molecular biologically how microRNA functions in the human brain due to drug addiction and how the microRNA is transferred to blood or serum by using an exosome as a means of transport. Therefore, the problem that, even though the change in the expression pattern of the microRNA in the brain is validated, it is difficult to apply the change in the expression pattern in the brain for clinical purposes directly and simply, was solved.

### TECHNICAL FIELD

The present disclosure relates to a biomarker for diagnosis of drug addiction and a kit for diagnosis of drug addiction.

### BACKGROUND ART

In general, a “drug (narcotic)” refers to compound having an effect similar to that of opium and its derivatives. By dictionary definition, it is defined as an opioid drug or an opium-like drug derived from the plant poppy, including addictive drugs such as morphine, cocaine, noscapine, papaverine, thebaine, heroin, etc. For a compound to be classified as a drug, it should be proven to be capable of inducing “significant emotional and behavioral changes”, inducing a state of “painless coma” and causing severe risks such as dependence, resistance and addiction.

Most drug addicts start using drugs for amusement, pleasure or pain killing. However, they are gradually addicted to the drugs and need stronger drugs. The common pathological symptoms of the drug addicts include fatigue, emotional lability, semi-coma, vomiting, etc. Because aggravated early symptoms can lead to antisocial and unethical behaviors and to social problems, diagnosis of drug addiction is of great importance.

Many researches are being conducted to elucidate the cause of these pathological symptoms. However, the efforts to elucidate the cause of emotional lability, etc. caused by drug addiction and resolve the cause and diagnose drug addiction are insufficient as yet.

A microRNA is a small RNA that regulates gene expression of living organisms. It is drawing attentions as it is found out that the microRNA binds to a mRNA complementarily and functions as a key regulatory factor of gene expression in cells.

However, there have not been many previous researches on molecular biological elucidation of the function of the microRNA in drug addiction and development of a biomarker for diagnosis of drug addiction or a therapeutic agent for drug addiction based thereon.

Despite the fact that the microRNA can be an important key in diagnosis of drug addiction because its expression pattern in the brain is changed due to drug addiction, there have been limitations in direct and convenient clinical applications because it is very difficult to extract it directly from the brain.

As a prior art document relating to the present disclosure, Korean Patent Registration No. 10-0715939 (paten document 1) relates to a therapeutic agent for drug addiction, which contains a Scutellaria baicalensis root extract, a betaine or a mixture thereof as an active ingredient and contains a pharmaceutically acceptable carrier. However, the paten document 1 neither discloses nor suggests a biomarker for diagnosis of drug addiction or a kit for diagnosis of drug addiction using a molecular biological method.

### REFERENCES OF RELATED ART

### Patent Documents

Korean Patent Registration No. 10-0715939.

### DISCLOSURE

### Technical Problem

The present disclosure is designed to solve the problems described above and is directed to providing a biomarker for diagnosis of drug addiction and a kit for diagnosis of drug addiction. In particular, it is directed to elucidating the cause and mechanism of abnormal behavior caused by drug addiction molecular biologically and providing a biomarker for diagnosis of drug addiction based thereon. The biomarker is provided using a microRNA and can be applied more directly and conveniently for clinical purposes. The present disclosure is also directed to providing a kit for diagnosis of drug addiction, which contains the biomarker.

### Technical Solution

In order to solve the problems described above, the present disclosure provides a biomarker for diagnosis of drug addiction, which contains microRNA-137 represented by SEQ ID NO 1 or its complementary base sequence represented by SEQ ID NO 2.

The expression pattern of the microRNA-137 in the human brain may be changed due to drug addiction, the microRNA-137 with the expression pattern changed in the brain may be transferred to blood or serum as being contained in an exosome and drug addiction may be diagnosed by extracting the microRNA-137 transferred to the blood or serum as being contained in the exosome.

The present disclosure also provides a kit for diagnosis of drug addiction, which contains a primer or a probe binding specifically to the biomarker, or an antibody binding specifically to a protein encoded by SEQ ID NO 1 or its complementary base sequence.

### Advantageous Effects

A biomarker for diagnosis of drug addiction and a kit for diagnosis of drug addiction according to the present disclosure can diagnose drug addiction by using a molecular biological method. Especially, the present disclosure has investigated molecular biologically how microRNA functions in the human brain due to drug addiction and how the microRNA is transferred to blood or serum by using an exosome as a means of transport. Therefore, the problem that, even though the change in the expression pattern of the microRNA in the brain is validated, it is difficult to apply the change in the expression pattern in the brain for clinical purposes directly and simply, was solved.

### BRIEF DESCRIPTION OF DRAWINGS

FIG. 1 shows a result of investigating the expression level of the miRNAs miR-137, -132 and -9 in the brain of normal mouse by fluorescence in situ hybridization.

FIG. 2 shows a result of measuring the change in the expression pattern of the miRNAs miR-137, -132 and -9 in the brain of a mouse into which cocaine has been injected repeatedly.

FIG. 3 shows the change in miR-137, -132 and -9 in the serum exosome of a methamphetamine-addicted patient and the sensitivity and specificity thereof.

FIG. 4 shows that the microRNA delivered to the serum exosome is derived from the brain.

FIG. 5 shows the change in microRNA-137, -132 and -9 in the cerebellum after final cocaine injection.

FIG. 6 shows the change in microRNA-137, -132 and -9 in the exosome-depleted serum (EDS) after final cocaine injection.

FIG. 7 shows the change in the expression pattern of the miRNAs miR-137, -132 and -9 in the brain of a mouse into which cocaine has been injected repeatedly.

FIG. 8 compares the expression pattern of three microRNAs (miR-137, -132 and -9) in the serum exosomes of methamphetamine-addicted patients depending on the period of time during which use of methamphetamine has been interrupted (less than 1 year vs 1 year or longer).

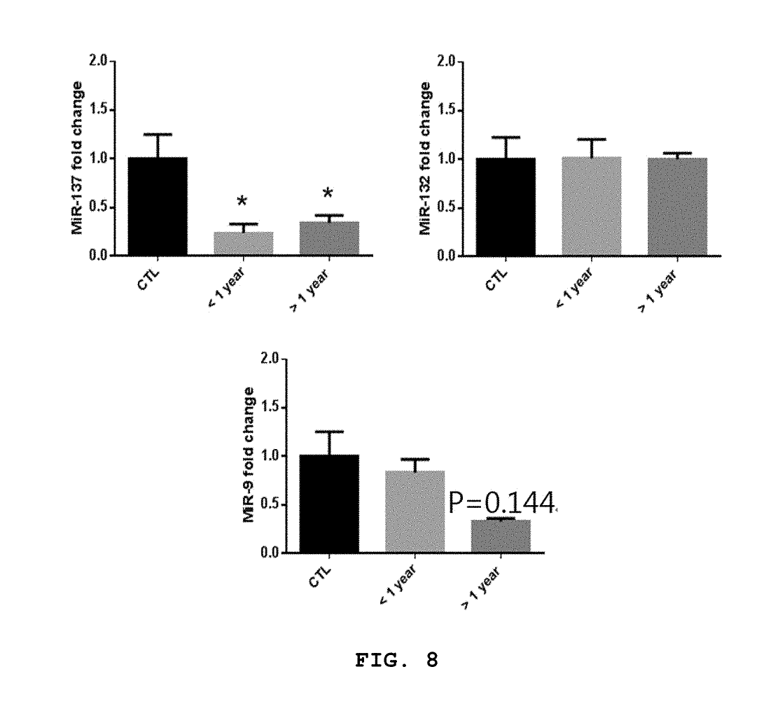

FIG. 9 compares the stability of microRNAs accumulated in exosomes under various conditions.

### BEST MODE

The inventors of the present disclosure have made efforts to develop a biomarker for diagnosis of drug addiction and a kit for diagnosis of drug addiction, which contains the same, by elucidating the cause of abnormal behavior caused by drug addiction molecular biologically. As a result, they have discovered a biomarker for diagnosis of drug addiction and a kit for diagnosis of drug addiction according to the present disclosure and have completed the present disclosure.

In general, early diagnosis of neuropsychiatric disorders including drug addiction is very important for effective treatment. In particular, when a drug such as a narcotic is administered, the brain responds first. The response leads to change in the expression pattern of specific genes or hormones in the brain. A microRNA (miRNA) is a short non-coding RNA containing 18-23 nucleotides and functions in regulation of transcription and silencing of messenger RNAs (mRNAs). It was recently reported that many miRNAs are tissue-specific and, thus, can be blood-based biomarkers. A large quantity of miRNAs are circulating in plasma or serum either freely or as being enclosed in exosomes. The exosomes are released from multivesicular bodies existing in cells as small vesicles (40-100 nm). Although miRNAs are mainly involved in the regulation of their cells, the miRNAs enclosed in exosomes are expected to be involved in cell-cell signaling.

Therefore, the inventors of the present disclosure expected that there would be specific microRNAs whose expression pattern changes significantly in the brain in case of drug addiction. Indeed, the inventors of the present disclosure have found out that the expression of the miR-137 disclosed in the present disclosure is significantly decreased in the brain in case of drug addiction (see ‘Test Example’). Despite the fact that the expression pattern of the particular microRNA in the brain is changed significantly in the brain due to drug addiction, it is very difficult to extract the microRNA directly from the brain tissue and use it for diagnosis or treatment of drug addiction. The inventors of the present disclosure have found out, in addition to the fact that the expression pattern of the specific microRNA (miR-137) in the brain tissue is changed due to drug addiction, that the specific microRNA (miR-137) is transferred from the brain to blood or serum by an exosome and can be extracted directly and conveniently from the blood or serum. As a result, the specific microRNA (exosomal miR-137) can be used to diagnose drug addiction. Hereinafter, the present disclosure is described in more detail.

Specifically, the biomarker for diagnosis of drug addiction according to the present disclosure contains microRNA-137 represented by SEQ ID NO 1 or its complementary base sequence represented by SEQ ID NO 2.

The expression pattern of the microRNA-137 in human brain is changed due to drug addiction. The microRNA-137 whose expression pattern is changed in the brain is transferred to blood or serum as being enclosed by an exosome. After being transferred to the blood or serum as being enclosed by the exosome, the microRNA-137 whose expression pattern is changed in the brain can be extracted from the blood or serum. Therefore, the microRNA-137 extracted from the blood or serum can be used as a biomarker for diagnosis of drug addiction without having to extract the microRNA-137 from the brain. That is to say, the microRNA-137 which is transferred to blood or serum as being enclosed by the exosome can be used as a biomarker capable of diagnosing drug addiction directly and conveniently in clinical applications.

The change in the expression pattern of the microRNA-137 in the brain may be a statistically significant one, although there is no particular limitation. The criterion of the statistical significance may be P<0.05. And, the change in the expression pattern may be increase or decrease of expression.

That is to say, the biomarker for diagnosis of drug addiction according to the present disclosure resolves the problem of difficult in extracting the microRNA whose expression pattern changes in the brain when diagnosing drug addiction using the microRNA. The present disclosure also relates to diagnosis of drug addiction using a molecular biological method.

The kit for diagnosis of drug addiction according to the present disclosure contains microRNA-137 represented by SEQ ID NO 1 or its complementary base sequence represented by SEQ ID NO 2, a fragment thereof, a primer or a probe binding specifically thereto, or an antibody binding specifically to a protein encoded thereby.

The probe binding specifically to the microRNA-137 represented by SEQ ID NO 1 may be a cDNA sequence represented by SEQ ID NO 2.

In an exemplary embodiment, the kit for diagnosis of drug addiction may contain a primer capable of specifically amplifying the microRNA-137 represented by SEQ ID NO 1, which is contained in any diagnostic kit known in the art without particular limitation. The amplification may be achieved through PCR. As the primer, Applied Biosystems's Cat. No. 4427975 (TaqMan MicroRNA Assay), Assay ID: 001129 may be used, for example. Also, a PCR mixture consisting of dNTP, a DNA polymerase and a buffer may be used for the PCR. In addition, the kit may contain various marker enzymes. In addition, a washing buffer may be used after the PCR and the reaction of the marker enzymes.

The kit for diagnosis of drug addiction may perform a first polymerase chain reaction (PCR) by adding a sample containing microRNA-137 onto a solid support of the kit for diagnosis of drug addiction together with the PCR mixture. Then, a second PCR may be performed by adding additional primers. Then, the support may be washed with a washing buffer after the PCR. Subsequently, the microRNA-137 may be analyzed using various marker enzymes and drug addiction may be diagnosed through this process.

The kit for diagnosis of drug addiction may contain the components of any kit known in the art. In a specific exemplary embodiment, the kit may contain:

a primer which amplifies the biomarker extracted from the sample;

a buffer and an enzyme for a polymerase chain reaction; and

a means for motion and separation which moves and separates the amplification product of the polymerase chain reaction according to size by microcapillary electrophoresis.

The means for motion and separation may include: a microcapillary electrophoresis chip wherein micrometer-sized channels and chambers are formed on a glass, plastic or silicon substrate; and a motion control means which is capable of controlling the motion of the microRNA-137 in the microcapillary electrophoresis chip via an electric field. The microRNA-137 moved and separated by the means for motion and separation may be attached with a fluorescent material for localization. With this configuration, several samples can be analyzed on a centimeter-sized chip. Therefore, it may be advantageous in terms of device miniaturization and cost reduction. In addition, automated diagnosis may be possible through the lab-on-a-chip technology whereby PCR, separation and detection are performed on a single chip. There is another advantage that diagnosis is possible only with a small quantity of sample.

### MODE FOR INVENTION

Hereinafter, the present disclosure will be described in detail through examples. However, the following examples are for illustrative purposes only and it will be apparent to those of ordinary skill in the art that the scope of the present disclosure is not limited by the examples.

### Example

The change in the expression pattern of the microRNA was observed around the striatum of a mouse which was repeatedly exposed to a narcotic such as cocaine, etc. and it was investigated whether the change in the expression pattern was observed also in blood or serum by the microRNA enclosed in an exosome. Moreover, it was observed whether the microRNA enclosed in the exosome had been transferred from the brain (striatum) to the periphery. In addition, it was investigated, suppose that the microRNA enclosed in the exosome reflected the change in the brain, whether it could be used as a reliable biomarker for diagnosis of drug addiction. Specifically, experiment was performed as follows.

Mice were intraabdominally administered with physiological saline or cocaine. After the final cocaine injection, the brain tissues (dorsal and ventral striatum, and cerebellum) and the serum exosome were sampled at different times. The change of the microRNA-137 in the brain and the exosome was analyzed by qRT-PCR. Lentivirus containing WPRE and copGFP fragments was used to determine whether the microRNA enclosed by the exosome in the serum originated from the brain tissue.

As a result, it was found out that the groups to which cocaine, etc. were injected repeatedly showed significant decrease in the microRNA-137 in the dST (dorsal striatum) and the exosome existing in the serum 7-28 days after the final exposure to cocaine. In addition, a strong correlation was found between the expression patterns in the dST (dorsal striatum) and the exosome existing in the serum. And, as a result of analyzing the exosome in the serum of patients addicted to methamphetamine, which is another psychostimulant as cocaine, it was confirmed that methamphetamine addicts could be distinguished with the exosomal microRNA-137 even after the use of methamphetamine had been stopped for 2.4 years on average. This result was significantly reliable with the AUC value of 0.8 or greater. In order to investigate whether the exosomal microRNA-137 reflects the change in the striatum in the brain, WPRE and copGFP which do not exist in the body were injected into the striatum of the brain and it was investigated whether they were observed in the exosome existing in the serum. Interestingly, the WPRE and copGFO were observed in the exosome existing in the serum. They were observed consistently in the striatum from 30 minutes until 24 hours after the injection of the WPRE and copGFP, and the mount of the WPRE and copGFO detected in the exosome existing in the serum began to increase significantly from 12 hours after the injection of the WPRE and copGFP. In addition, it was confirmed that the exosomal microRNA-137 was stable despite extreme temperatures and chemical treatments as compared to the microRNA not enclosed in the exosome.

Through these results, it was confirmed that the exosomal microRNA-137 existing in the serum can be used as a biomarker that sensitively diagnoses drug addiction to psychostimulants such as cocaine, etc. In addition, it was confirmed through these results that drug addiction can be diagnosed simply by extracting the microRNA from the serum without having to extract it directly from the brain. Therefore, drug addiction can be diagnosed more directly and conveniently in clinical applications. Hereinafter, the procedures leading to these results are described in more detail.

<Selection of Mice Used for Experiments>

Male C57BL/6J mice (Daehan Biolink, Korea) weighing 21-24 g were used in all the following experiments. The mice were divided into several groups and housed in clear plastic cages with metal wire lids (3-5 mice per cage). Lighting was controlled with 12-hour cycles (lights on 7 am) and food and water were supplied. All the procedures of the experiments using the mice were conducted according to the guideline of the Institution Animal Care and Use Committee serving the Korea Institute of Science and Technology.

<Fluorescence In Situ Hybridization (FISH)>

In order to inhibit RNase activity and wash the tissue, the tissue was immersed in a DEPC-PBS solution for 5 minutes. For deactivation of RNase and fixation of tissue components including RNA, the tissue was fixed in a 4% buffered paraformaldehyde solution for about 10 minutes and then incubated with a proteinase K solution at room temperature for 20 minutes in order to degrade the proteins in the tissue. After conducting acetylation in 0.1 M TEA+0.25% acetic acid for 10 minutes, the resulting hybridization solution was heated sufficiently in a water bath at 50° C. The hybridization solution was mixed with a DIG-labeled RNA probe. The RNA probe mixture was placed on a slide, covered with HybriSlip (PGC, 62-6504-04, Frederick, Md., U.S.A.) or a plastic cover and incubated on a heat plate of about 55° C. for about a day. In a moist room, the slide was placed on a heat plate and was sealed hermetically after moistening with water using a gauze. The next day, the slide was taken out of the incubator and then incubated in a water bath at 50° C. for 10 minutes using a 2×SSC-50% formamide solution. After the hybrid slip was removed and the slide was incubated again in a water bath at 50° C. for 20 minutes using a 2×SSC-50% formamide solution, followed by incubation in a water bath at 50° C. for 10 minutes and incubation at room temperature for 10 minutes. When sufficient miRNA expression was expected, incubation was conducted with an RNase/NTE buffer in a water bath at 37° C. for 20 minutes. Then, incubation was conducted with an anti-DIG antibody (1:1000 dilution in DIG 1 solution, Roche, 1-093-274, Mannheim, Germany) at room temperature for 2 hours. Then, the DIG-binding signal was amplified using the TSA plus kit and observed under a microscope.

<Treatment with Drug and Cocaine>

Cocaine hydrochloride (MacFarlan Smith Ltd., UK) was dissolved in physiological saline (0.9% w/v). For a repeatedly administered cocaine group (repeated group), 20 mg/kg cocaine was injected intraabdominally once a day for 7 days. For a control group (saline group), physiological saline was injected intraabdominally once a day for 7 days. For an acutely administered cocaine group (acute group), physiological saline was injected intraabdominally for 6 days and cocaine was injected on day 7.

<Blood Sampling from Drug-Addicted Patients>

Blood samples of methamphetamine-addicted patients were supplied from the Addiction Brain Center of Euilji University Gangnam Euilji Hospital. People with similar ages with no smoking history were selected as a control group. All the experiments were conducted under consent and under approval from the Korea Institute of Science and Technology and Bugok National Hospital.

The information about the methamphetamine-addicted patients is given in Table 1 (CTL: age-matched controls).

TABLE 1

| | | | Duration | Abstinence |
|---|---|---|---|---|
| Group | Gender | Age | (year) | (year) |
| Patients (n = 20) | Male | 48.9 ± 1.8 | 21.4 ± 1.8 | 2.4 ± 0.6 |
| CTL (n = 11) | Male | 47.1 ± 2.3 | — | — |

<Preparation of Serum and Brain Tissue>

The mice were sacrificed by cervical dislocation 2 hours, 24 hours, 7 days, 14 days and 28 days after the final drug injection and whole blood was obtained by venipuncture. For the methamphetamine-addicted patients, the acquired blood samples were used. After separating serum from the blood by keeping at room temperature for 45 minutes, centrifugation was performed with a speed of 1,500×g at 4° C. for 20 minutes. Each serum sample was transferred to a 1.5-mL tube and was used immediately for exosome isolation or RNA extraction. Brain tissues were isolated immediately after the sacrificing of the animals and frozen at −80° C. Then, the frozen sections were cut to a thickness of 100 μm at −20° C. using a cryostat microtome (CM3050S, Leica) to obtain the sections of the dorsal striatum (dST), the ventral striatum (vST) and the cerebellum (Cbl).

<Exosome Isolation>

Exosomes were isolated from 100 μL of the serum using the commercially available exosome precipitation reagent ExoQuick™ (System Biosciences Inc., Mountain View, Calif., USA) according to the manufacturer's instructions. In brief, after adding to the sample the ExoQuick solution corresponding to 1/4 of the sample volume, the resulting mixture was stored at 4° C. The next day, the mixture was centrifuged with a speed of 1,500×g for 30 minutes and exosomes were obtained by removing the supernatant. The obtained exosomes were subjected to RNA extraction, protein expression analysis (western blot) or transmission electron microscopic (TEM) analysis. For the RNA extraction and the protein expression analysis, the mixture was mixed with 10 μL of cold phosphate-buffered saline (PBS). For the transmission electron microscopic analysis, the mixture was mixed with 100 μL of cold phosphate-buffered saline.

<TEM (Transmission Electron Microscopy)>

The isolated exosomes were fixed with 1% (v/v) glutaraldehyde at room temperature (RT) for 1 hour and applied on formvar-coated 200-mesh copper grids (ProSciTech, Queensland, Australia). They were kept at RT in dry state. The grids were washed twice with water for 5 minutes. Then, staining was performed with 1% (v/v) uranyl acetate for 10 minutes. Images were acquired by applying an acceleration voltage of 200 kV using the Gatan UltraScan 1000 (2×2k) CCD (charge-coupled device) camera coupled to the Tecnai F30 (FEI, Netherlands) electron microscope. The stains occurring on 10 different areas of the grids were captured. Imaging analysis was performed using the Image J software 1.43u (http://rsbweb.nih.gov/ij). After image scale setting, the size of individual vesicles was measured as diameter using the Image J software. The incomplete vesicles located at the edge portions of the image were excluded.

<Quantification of Mature miRNA by qRT-PCR>

The expression of mature microRNA was conducted following reverse-transcriptase polymerase chain reaction using specific RT primers (TaqMan MicroRNA Assay, Applied Biosystems, Cheshire, UK). 50 ng of total RNAs from the sample were used for the preparation of cDNAs. The cDNAs were amplified by quantitative real-time polymerase chain reaction (qRT-PCR) using Universal TaqMan Mix (with no AmpErase UNG) and miRNA-specific primers (ABI) according to the manufacturer's protocol. The reaction was conducted on the CFX Connect (BioRad, Reinach, Switzerland). All the reaction was performed in triplicates. The relative ratio of each target was calculated together with small nuclear RNA for brain tissues and serum miRNA-16 for normalization. The two groups were compared based on their 2-ddCt values and statistical significance was calculated by one-way analysis of variance (ANOVA). In addition, the Dunnett's post hoc test was performed with the statistical significance of P<0.05.

Information about the LNA (locked nucleic acid) probe sequences and DNA melting temperatures is given in Table 2.

TABLE 2

| LNA probe | Sequence (5′→3′) | Tₘ (° C.) |
|---|---|---|
| miR-137 | CTACGCGTATTCTTAAGCAATAA | 67 |
| miR-132 | CGACCATGGCTGTAGACTGTTA | 76 |
| miR-9 | TCATACAGCTAGATAACCAAAGA | 74 |
| miR-122 | AAACACCATTGTCACACTCCA | 76 |
| Scrambled | GTGTAACACGTCTATACGCCCA | 78 |

<WPRE Lentiviral Constructs>

pCDH-CMV-MCS-EF1-copGFP was purchased from System Biosciences. It includes WPRE (woodchuck posttranscriptional regulatory element) under the control of the cytomegalovirus promoter and a copGFP reporter.

<Intrastriatal Injection>

For intrastriatal administration of a lentiviral vector, the mouse was anesthetized with intraperitoneally injected ketamine-xylazine (120 and 6 mg/kg). Then, the mouse was placed on a stereotaxic frame (Kopf Instruments, USA). Three viral suspension injections (0.5 μL per injection; viral suspension concentrations ranged from 2.5×10⁷ to 3.0×10⁷ infectious units (IFU) per mL) were delivered to each side of the striatum per mouse. The injections were performed at 1.10 mm from the bregma; mediolateral (ML), ±1.00 mm from midline; dorsoventral (DV), −3.0, −2.5 and −2.0 mm below the dura.

<Stability of Exosomal miRNA>

The samples were stored for 24 hours at 4° C. or 37° C. For complete lysis of exosomes, Triton X-100 (Sigma) was added to a final concentration of 1%. Then, the exosomes were incubated with RNase A (Sigma, St. Louis, Mo., USA) at a final concentration of 10 μg/MI for 30 minutes at RT.

<Statistical Analysis>

Statistical analysis was conducted by the Dunnett post hoc test and the unpaired t-test or one-way analysis of variance (ANOVA) using the GraphPad Prism (V6.0, GraphPad, San Diego, Calif., USA). The Pearson coefficients were calculated to test correlation. In all the tests, P<0.05 was used as a measure of statistical significance.

### Test Example

<microRNA Expression in Brain of Normal Mice Observed by Fluorescence In Situ Hybridization>

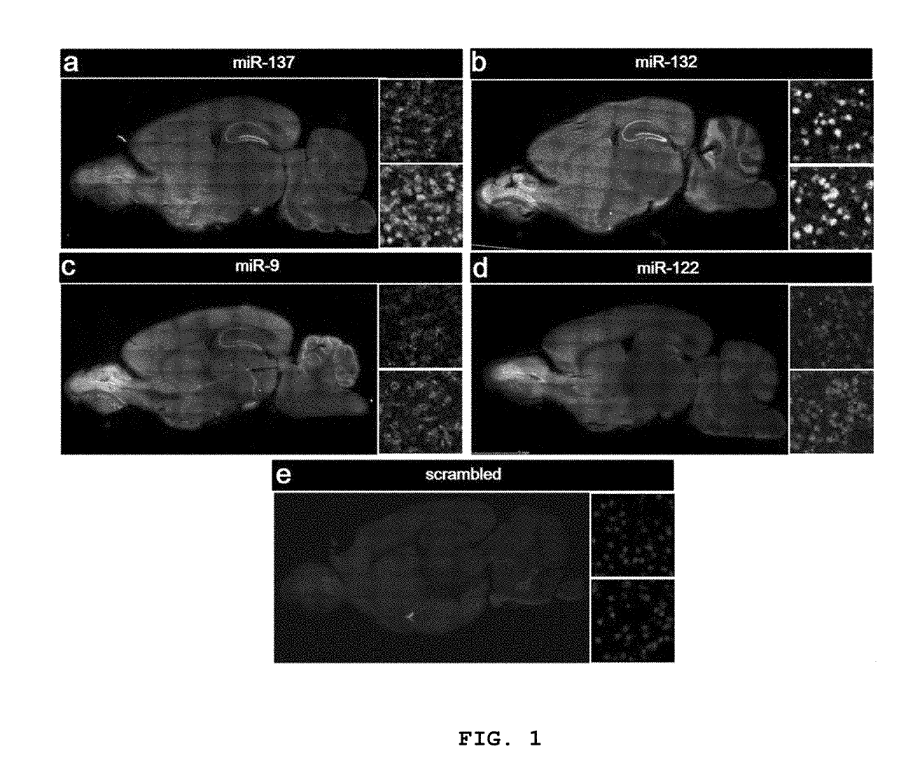

Fluorescence in situ hybridization was conducted to investigate the expression level of microRNAs in normal brain. The result is shown in FIG. 1. The fluorescence in situ hybridization was conducted using miR-137, -132 and -9, which are known to be distributed in large quantities in the brain, and miR-122 which is known to be hardly expressed in the brain. In particular, the striatum (ST) was observed intensively because it is known to be associated with drug addiction. miR-137 was expressed moderately in the dorsal striatum (dST) and strongly in the ventral striatum (vST) (FIG. 1 a). miR-132 was expressed strongly both in the dST and the vST (FIG. 1 b). miR-9 was expressed relatively weakly and was observed to be expressed more strongly in the olfactory bulb, the hippocampus, the cerebellum, etc. than in the vST or the dST (FIG. 1 c). miR-122, which is known to be expressed in large quantities in the liver and is hardly expressed in the brain, was hardly expressed in the brain as expected (FIG. 1 d). The scramble used as a negative control group was not expressed in the brain (FIG. 1 e).

<Change in MiRNA-137 Expression Pattern in Brain and Exosome after Repeated Cocaine Iniection>

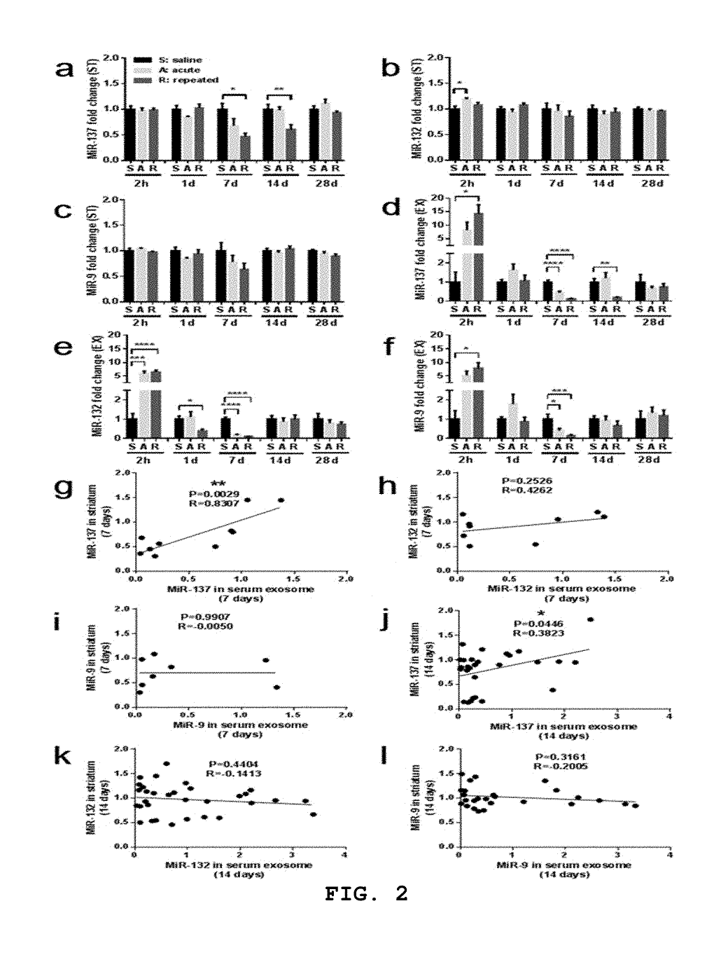

The change in the expression pattern of the miRNAs miR-137, -132 and -9 in the brain was measured after repeated cocaine injection. The result is shown in FIG. 2.

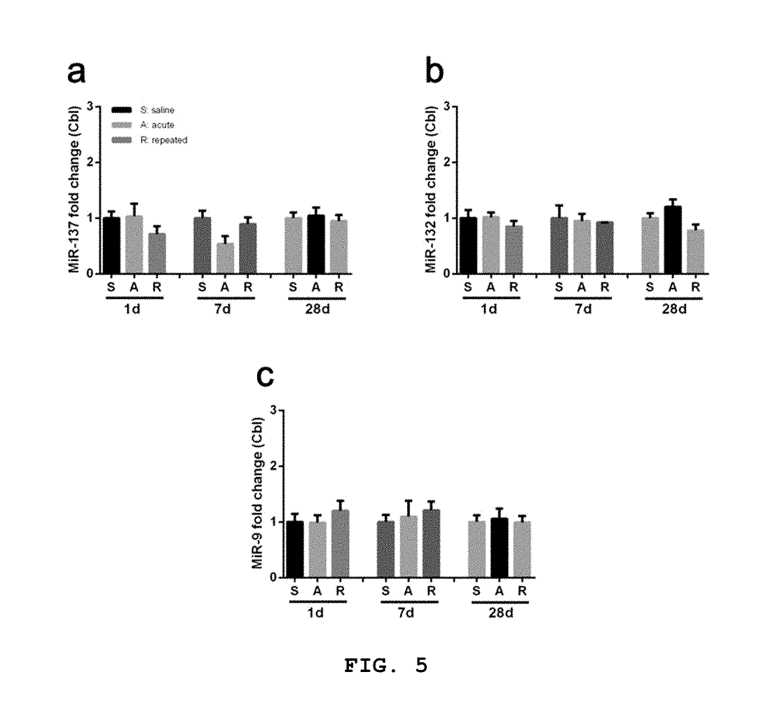

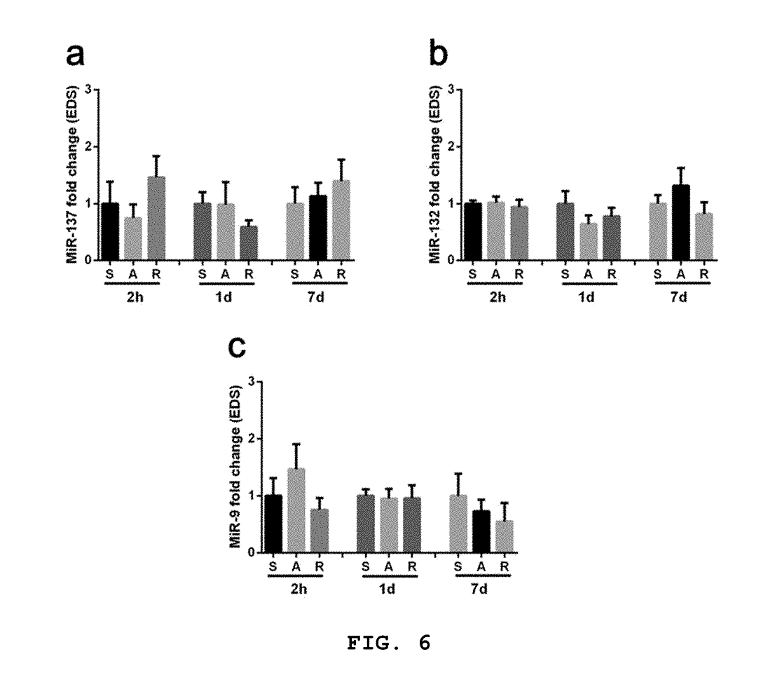

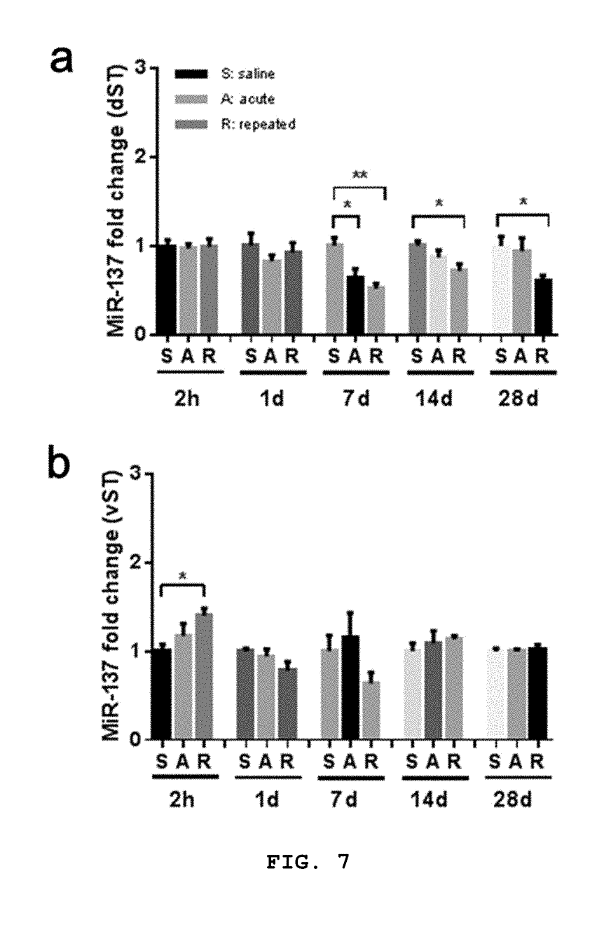

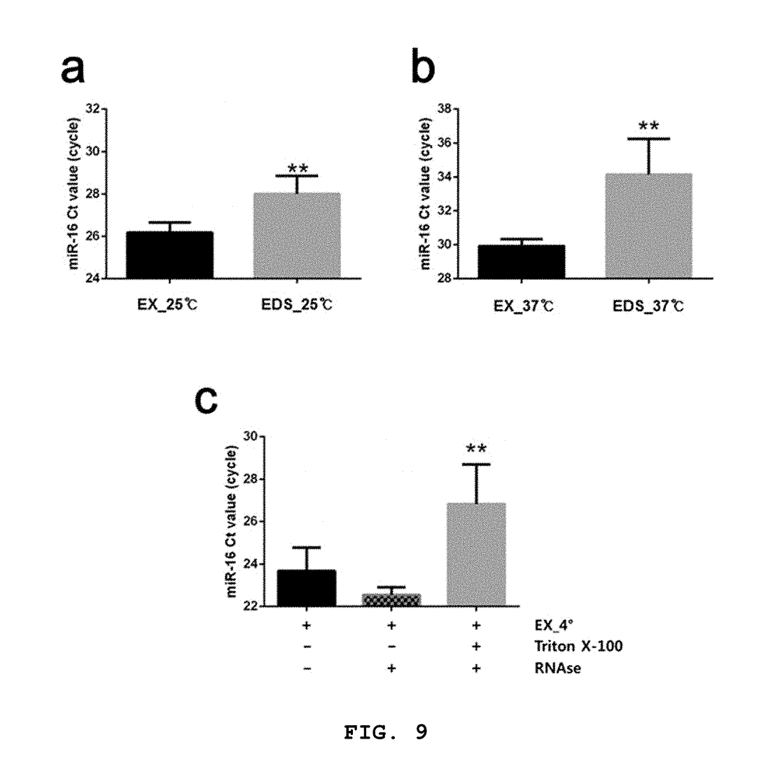

As seen from FIG. 2, significant change (in FIG. 2, significant change was denoted by ‘*’) of miR-137 was observed in the ST after 7-28 days (FIG. 2 a). However, no significant change was observed for miR-137 after 2 hours or 24 hours (FIG. 2 a). For miR-132, significant change was observed 2 hours after the cocaine injection. For miR-9, no significant change was observed. When additional experiments were performed for the ST (dST and vST) under the same conditions, miR-137 expression was decreased significantly in the dST on 7, 14 and 28 days after the final cocaine injection. But, in the vST, increase was observed after 2 hours only for the repeated group (FIG. 7 a, b). Through these results, it was confirmed that only the miR-137 shows significant change in expression pattern in the brain after repeated cocaine injection unlike other miRNAs. When additional experiments were performed for the cerebellum, it was confirmed that the change in the expression level of miR-137, -132 and -9 is specific for the ST which is particularly associated with drug addiction (FIG. 5). In addition, in order to compare the expression pattern of the microRNAs in the constituents of blood, serum was separated into exosomes (EX) and exosome-depleted serum (EDS). The quantity of mature miR-137, -132 and -9 was compared by calculating the Ct value by qRT-PCR. Surprisingly, the miR-137, -132 and -9 that were detected with significant quantities in the brain tissue were observed in the EX to such a level as might be thought in response to exposure to cocaine (Ct values of between 25 and 35, data not shown) (FIG. 2 d-f). All the miR-137, -132 and -9 were remarkably increased until 2 hours after the cocaine injection and the miR-132 was decreased significantly from day 1 until day 7. Differently from this, the miR-137 showed remarkable decrease from day 7 until day 28 after the cocaine injection. The miR-9 showed significant decrease only on day 7 after the cocaine injection (FIG. 2 d-f). In conclusion, whereas significant decrease of miR-137 was detectable for the repeated group 7-28 days after the final injection, there was no significant change in the expression of miR-132 or miR-9 from 14 days after the final injection (FIG. 2 e-f). However, this change was not observed in the EDS (FIG. 6). Additionally, as a result of comparing the stability of miRNA transfer for the EX and the EDS, it was confirmed that the miRNA is more stable in the EX (FIG. 9).

Based on these results, the correlation of miR expression in the ST and the EX was investigated (FIG. 2 g-l). As a result, it was confirmed that the expression of miR-137 only is associated with the ST (FIG. 2 g, j).

<Change of miR-137 in Serum of Methamphetamine-Addicted Patients>

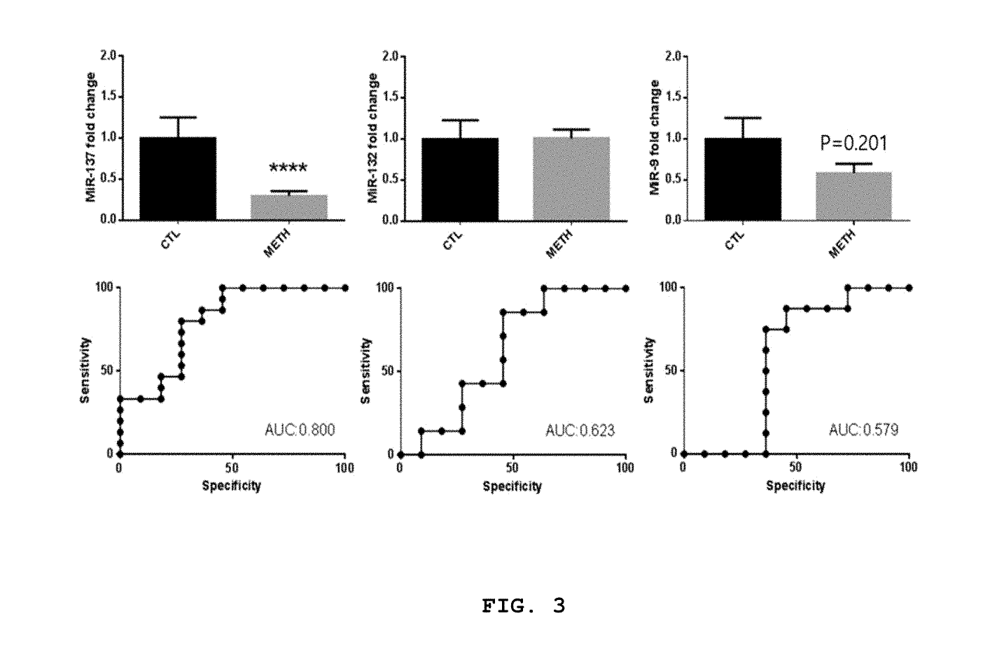

In order to investigate whether the change in miR-137 in response to the drug is also found in the exosome isolated from the serum of a drug-addicted patient, the serum samples of methamphetamine-addicted patients were examined (FIG. 3). As a result, it was found out that miR-137 expression was remarkably decreased in the exosomes of methamphetamine-addicted patients (FIG. 3 a). However, no change was observed for miR132 and miR-9 (FIG. 3b, c). When this change was compared with the age-matched control group, it was found out that only miR-137 shows strong sensitivity and specificity (FIG. 3 d-f). These results demonstrate that miR-137 is a very reliable indicator for diagnosis of drug addiction.

<Brain-Derived Exosomal microRNA-137 is Detectable in Serum>

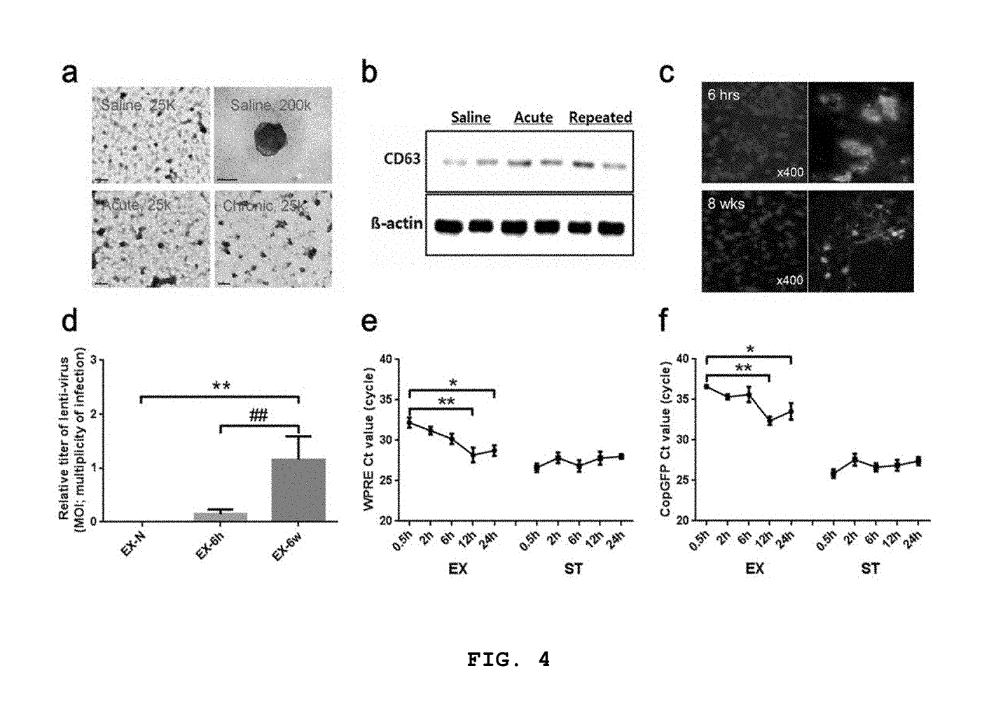

The exosomes extracted from the serum were examined by TEM and by using exosome markers (FIG. 4 a, b). Also, in order to investigate whether the exosomes extracted from the serum were transferred from the brain tissue, a lentiviral vector was used to deliver WPRE fragments to the dST. The lentiviral vector including dST WPRE and copGFP reporters was infused using a stereotaxic device. As a result, the expression of the injected coGFP in the dST was confirmed by a histochemical method (FIG. 4 c). The expression of WPRE and copGFP was increased in the brain tissue and the exosome 12 and 24 hours after the lentiviral vector infusion (FIG. 4 d, e). In addition, it was observed that the WPRE existing in the exosome was stably expressed until 6 hours (FIG. 4 f). Interestingly, WPRE does not occur naturally in mammals. Therefore, the WPRE detected in the serum suggests that the exosome is derived from the brain tissue. This result confirms that biomolecules such as microRNA were delivered from the brain tissue to blood via exosomes.

Although specific examples of the present disclosure were described above, the present disclosure can be changed variously within the scope of the present disclosure without being limited thereto. It is obvious that such changes belong to the scope of the appended claims.

What is claimed is:

1. A biomarker for diagnosis of drug addiction, comprising microRNA-137 represented by SEQ ID NO 1 or its complementary base sequence represented by SEQ ID NO 2.

2. The biomarker for diagnosis of drug addiction according to claim 1, wherein the expression pattern of the microRNA-137 in the human brain is changed due to drug addiction, the microRNA-137 with the expression pattern changed in the brain is transferred to blood or serum as being contained in an exosome and drug addiction is diagnosed by extracting the microRNA-137 transferred to the blood or serum as being contained in the exosome.

3. A kit for diagnosis of drug addiction, comprising microRNA-137 represented by SEQ ID NO 1 or its complementary base sequence represented by SEQ ID NO 2, a fragment thereof, a primer or a probe binding specifically thereto, or an antibody binding specifically to a protein encoded thereby.


SEQUENCE LISTING (1 sequence)

SEQ ID NO: 1 - RNA, 23 nt, Unknown
  micro RNA-137
uuauugcuuaagaauacgcguag